(19) United States
(12) Patent Application Publication
(10) Pub. No.: US 2025/0177625 A1
(43) Pub. Date: Jun. 5, 2025

(54) FILTERING DEVICES, SUCH AS FOR USE WITH CLOT TREATMENT SYSTEMS, AND ASSOCIATED SYSTEMS AND METHODS

(71) Applicant: Inari Medical, Inc., Irvine, CA (US)

(72) Inventor: Benjamin Edward Merritt, San Clemente, CA (US)

(21) Appl. No.: 18/963,471

(22) Filed: Nov. 27, 2024

Related U.S. Application Data
(60) Provisional application No. 63/605,188, filed on Dec. 1, 2023.
(60) Provisional application No. 63/697,970, filed on Sep. 23, 2024.

(51) Int. Cl. A61M 1/34; A61M 39/24

(52) U.S. Cl. CPC A61M 1/34; A61M 39/24; A61M 2039/2426; A61M 2205/7545

(57) ABSTRACT

Disclosed herein are filtering devices for use with clot treatment systems, and associated systems and methods. In some embodiments, a filtering device, includes a body, a filter, a collection component, and an outlet conduit. The filter can be coupled to the body and configured to receive material and permit a portion of the received material to pass therethrough. The collection component can be positioned within the body to receive the portion of the material that passes through the filter. The outlet conduit can be operably coupled to the collection component and configured to allow the portion of the material to be removed from the chamber.

### CROSS-REFERENCE TO RELATED APPLICATION

The present application claims priority to U.S. Provisional App. No. 63/605,188; filed on Dec. 1, 2023, and titled “FILTERING DEVICES, SUCH AS FOR USE WITH CLOT TREATMENT SYSTEMS, AND ASSOCIATED SYSTEMS AND METHODS”; and to U.S. Provisional App. No. 63/697,970; filed on Sep. 23, 2024, and titled “FILTERING DEVICES, SUCH AS FOR USE WITH CLOT TREATMENT SYSTEMS, AND ASSOCIATED SYSTEMS AND METHODS”; the entireties of which are hereby incorporated by reference herein.

### TECHNICAL FIELD

The present technology generally relates to clot treatment systems, including filtering devices for use with clot treatment systems, and associated systems and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

Various systems exist for performing a thrombectomy or removing occlusive material to reestablish blood flow within a patient. Such devices can remove the target clot material and often additionally remove one or more fluids (e.g., blood) along with the clot material. These devices are often designed to make it difficult to return removed fluids (e.g., blood) to the patient, even when doing so may be advantageous. For example, some existing devices include bypass circuits that can be cumbersome to set up and may require a specially trained user (e.g., a perfusionist) present to operate correctly.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system configured in accordance with embodiments of the present technology.

FIG. 2 is a perspective view of a filtering device of the clot treatment system of FIG. 1 in accordance with embodiments of the present technology.

FIGS. 3A and 3B are perspective views of the filtering device of FIG. 2 in accordance with embodiments of the present technology.

FIG. 4 is a perspective view of a filtering device configured in accordance with additional embodiments of the present technology.

FIG. 5 is a perspective view of a filtering device configured in accordance with embodiments of the present technology.

FIG. 6A is a perspective view of a filtering device configured in accordance with additional embodiments of the present technology.

FIG. 6B is a partially-exploded perspective view of the filtering device of FIG. 6A in accordance with embodiments of the present technology.

FIG. 6C is a perspective cross-sectional view of the filtering device of FIGS. 6A and 6B in accordance with embodiments of the present technology.

FIG. 7 is a flow diagram of a method for removing clot material from a patient, in accordance with additional embodiments of the present technology.

FIG. 8 is a partially schematic side view of a clot treatment system configured in accordance with additional embodiments of the present technology.

FIG. 9 is a flow diagram of a method for removing clot material from a patient in accordance with additional embodiments of the present technology.

### DETAILED DESCRIPTION

The present technology is generally directed to filtering devices for use in clot treatment systems, and associated systems and methods. In some embodiments, a filtering device in accordance with the present technology can include a body defining a chamber, a filter, a collection component, and an outlet conduit. The filter can be coupled to the body and configured to receive material, such as clot material aspirated from a patient, and permit a portion of the received material, such as blood, to pass therethrough and, e.g., into the chamber of the body. In some embodiments, the chamber can be sized to hold material from repeated aspirations, which is expected to allow a user to perform repeated aspirations without emptying the filtering device. The collection component can be positioned within the chamber to receive the portion of the material that passes through the filter. The outlet conduit can be operably coupled to the collection component and configured to allow the portion of the material to be removed from the chamber. For example, a pressure source such as a syringe can be operably coupled to the outlet conduit and actuated to draw filtered material (e.g., blood) from within the chamber. In some embodiments, a fluid control device such as a valve is operably coupled to the outlet conduit to selectively allow or prevent the filtered material from leaving the chamber.

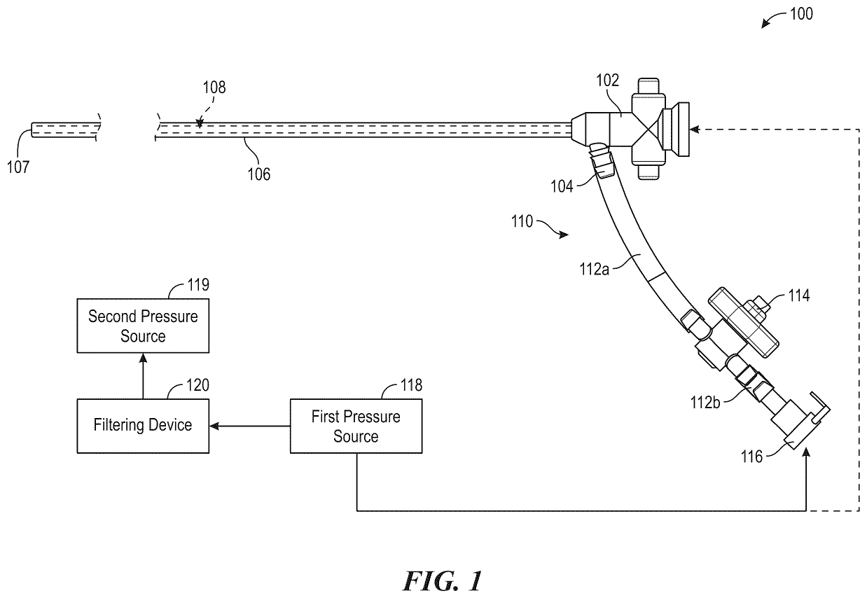

Certain details are set forth in the following description and in FIGS. 1-9 to provide a thorough understanding of various embodiments of the present technology. In other instances, well-known structures, materials, operations, and/or systems often associated with intravascular procedures, clot removal procedures, clot treatment systems, clot treatment devices, fluid control devices, syringes, blood filters, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, tubing assembly 110 is first introduced and discussed with reference to FIG. 1.

To the extent any materials incorporated herein by reference conflict with the present disclosure, the present disclosure controls.

FIG. 1 is a partially schematic side view of a clot treatment system 100 (“the system 100”) configured in accordance with embodiments of the present technology. The system 100 can also be referred to as an aspiration assembly, a vascular access system, a clot removal system, a thrombectomy system, and/or the like. In the illustrated embodiment, the system 100 includes a tubing assembly 110 fluidly coupled to a catheter 106 via a valve 102. In some embodiments, the catheter 106 is an elongate member (e.g., a sheath, a shaft) configured to be inserted into and through a patient's vasculature and used to, for example, remove or otherwise treat clot material therein. In other embodiments, the catheter 106 can be an introducer sheath configured to be inserted through the skin and tissue tract of the patient to provide an access site through which other components (e.g., other catheters used to treat clot material) can traverse to easily access the vasculature. Accordingly, while referred to as “catheter 106,” the catheter 106 can comprise an introducer sheath, an access sheath, and/or another type of elongate member configured to be inserted through the skin and tissue tract and/or to traverse the vasculature of a patient. The catheter 106 can be a large bore catheter, having, for example, a size equal to or greater than 16 French (Fr), such as 20 Fr, 22 Fr, 24 Fr, 26 Fr, 28 Fr, 30 Fr, 32 Fr, and/or the like. In general, the system 100 (i) can include features generally similar in structure and/or function, or identical in structure and/or function, to those of the clot treatment systems described in detail in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

The catheter 106 further defines a lumen 108 (shown in dashed line in FIG. 1) extending entirely therethrough, e.g., from the valve 102 to a distal terminus 107 of the catheter 106. The lumen 108 is not necessarily shown to scale in FIG. 1 and can have a diameter close to the outer diameter of the catheter 106. That is the catheter 106 can have a relatively thin wall. The catheter 106 can have varying lengths, flexibilities, shapes, thicknesses, and/or other properties along its length. For example, the catheter 106 can comprise one or more coils, braids, and/or other structures positioned between one or more liner layers (e.g., an inner liner layer and an outer liner layer). In some embodiments, the catheter 106 can include several features generally similar or identical in structure and/or function to any of the catheters described in (i) U.S. patent application Ser. No. 17/529,018, titled “CATHETERS HAVING SHAPED DISTAL PORTIONS, AND ASSOCIATED SYSTEMS AND METHODS,” and filed Nov. 17, 2021, (ii) U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, (iii) U.S. patent application Ser. No. 18/159,507, titled “ASPIRATION CATHETERS HAVING GROOVED INNER SURFACE, AND ASSOCIATED SYSTEM AND METHODS,” and filed Jan. 25, 2023, and/or (iv) U.S. patent application Ser. No. 18/463,960, titled “CATHETERS HAVING MULTIPLE COIL LAYERS, AND ASSOCIATED SYSTEMS AND METHODS,” and filed Sep. 8, 2023, each of which is incorporated by reference herein in its entirety.

The valve 102 is fluidly coupled to the lumen 108 of the catheter 106 and can be integral with or coupled to the catheter 106 such that these components move together. In some embodiments, the valve 102 is a hemostasis valve that is configured to maintain hemostasis during a clot treatment procedure by preventing fluid flow in a proximal direction through the valve 102 as various components such as dilators, delivery sheaths, pull members, guidewires, interventional devices, other aspiration catheters, and so on are inserted through the valve 102 to be delivered through the catheter 106 to a treatment site in a blood vessel. The valve 102 can include a branch or side port 104 configured to fluidly couple the lumen 108 of the catheter 106 to the tubing assembly 110. In some embodiments, the valve 102 can be a valve of the type disclosed in U.S. patent application Ser. No. 16/536,185, titled “HEMOSTASIS VALVES AND METHODS OF USE,” and filed Aug. 30, 2018, which is incorporated herein by reference in its entirety.

In the illustrated embodiment, the tubing assembly 110 fluidly couples the catheter 106 to a first or primary pressure source 118 (“pressure source 118”). The pressure source 118 can be a syringe, and more particularly can be a syringe including some features generally similar in structure and/or function, or identical in structure and/or function, to those of any of the syringes described in U.S. patent application Ser. No. 17/396,426, titled “AUTOMATICALLY-LOCKING VACUUM SYRINGES, AND ASSOCIATED SYSTEMS AND METHODS,” and filed Aug. 8, 2021, and/or in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” and filed Mar. 30, 2023, each of which is incorporated by reference herein in its entirety. Additionally, or alternatively, the pressure source 118 can include an electric pump and/or one or more other suitable pressure sources. In these and/or other embodiments, the pressure source 118 can be configured to generate (e.g., form, create, charge, build-up) a vacuum (e.g., negative relative pressure) and store the vacuum for subsequent application to the catheter 106.

The tubing assembly 110 can include one or more tubing sections 112 (individually labeled as a first tubing section 112a and a second tubing section 112b), at least one valve or fluid control device 114, and at least one connector 116 (e.g., a Toomey tip connector) for fluidly coupling the tubing assembly 110 to the pressure source 118 and/or other suitable components. In some embodiments, the connector 116 is a quick-release connector (e.g., a quick disconnect fitting) that enables rapid coupling/decoupling of the catheter 106 and the fluid control device 114 to/from the pressure source 118. The tubing assembly 110 and the catheter 106 can have a same or substantially same inner dimension to, for example, define a lumen or flow path of uniform or substantially uniform diameter extending from the distal terminus 107 of the catheter to the pressure source 118. In some embodiments, the fluid control device 114 is fluidly coupled to (i) the side port 104 of the valve 102 via the first tubing section 112a and (ii) the connector 116 via the second tubing section 112b. The fluid control device 114 is externally operable by a user to regulate the flow of fluid therethrough and, specifically, from the lumen 108 of the catheter 106 to the pressure source 118. For example, the fluid control device 114 can be transitioned between (i) a first or closed configuration in which the fluid control device 114 inhibits or even prevents fluid flow therethrough and (ii) a second or open configuration in which fluid can flow through the fluid control device 114.

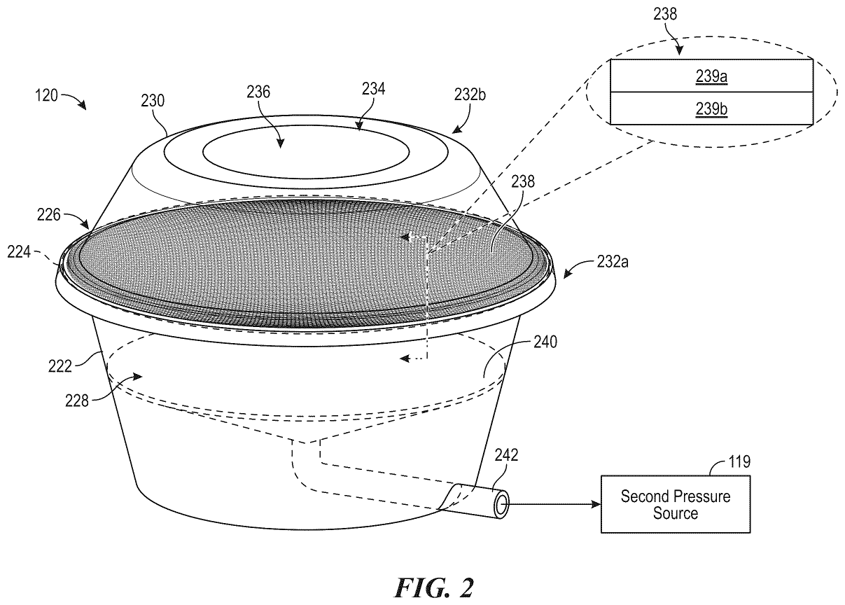

In some embodiments, the system 100 further includes a filtering device 120 and a second or secondary pressure source 119. The filtering device 120 can be configured to receive material (e.g., emboli, clot material, blood, other fluid, etc.) aspirated from the patient via the catheter 106 and to filter or otherwise separate at least a portion of the blood from this aspirated material. The secondary pressure source 119 can be operably coupled to (e.g., in fluid communication with) the filtering device 120 and configured to receive all, or at least a portion, of the filtered blood from the filtering device 120. Once received, the filtered blood can be reintroduced into the patient using the secondary pressure source 119. The filtering device 120 and the secondary pressure source 119 are described in greater detail below with reference to one or more of FIGS. 2-9.

During a clot removal procedure, at least a portion of the system 100, including at least a portion of the catheter 106, can be inserted through the vasculature of a patient to treat clot material therein. In some embodiments, the system 100 is inserted to a target treatment location proximate to the clot material through an introducer sheath that traverses the skin and tissue of the patient to provide an access site. The fluid control device 114 can be in the closed position during insertion of the catheter 106. After positioning the catheter 106 at the treatment location and with the fluid control device 114 in the closed position, a user/operator can generate a vacuum in the pressure source 118 by, for example, fluidly coupling the pressure source 118 to the connector 116 and withdrawing a plunger of the pressure source 118. In this manner, a vacuum is charged within the pressure source 118 (e.g., a negative pressure is maintained) before the pressure source 118 is fluidly connected to the lumen 108 of the catheter 106 (e.g., by opening the fluid control device 114).

To aspirate the lumen 108 of the catheter 106, the user can actuate (e.g., open) the fluid control device 114 to fluidly connect the pressure source 118 to the catheter 106 and thereby apply or release the vacuum stored in the pressure source 118 to the lumen 108 of the catheter 106. Opening of the fluid control device 114 instantaneously or nearly instantaneously applies the stored vacuum pressure to the tubing assembly 110 and the catheter 106, thereby generating a suction pulse throughout the catheter 106 that can aspirate the clot material into the catheter 106. In some embodiments, the vacuum from the pressure source 118 is applied with the fluid control device 114 in an open position (e.g., to provide continuous vacuum). That is, the user can generate the vacuum in the pressure source 118 while the fluid control device 114 is open (e.g., while the pressure source 118 is fluidly connected to the lumen 108 of the catheter 106) to thereby aspirate the clot material while also simultaneously generating the vacuum—that is, for example, without or substantially without storing the vacuum in the pressure source 118.

Material aspirated via the catheter 106 can be received within the pressure source 118. For example, if the pressure source 118 includes a syringe, the aspirated material can be received within a barrel of the syringe. In these and/or other embodiments, the aspirated material can transferred to the filtering device 120 which can filter or otherwise separate blood from the other aspirated material. For example, the pressure source 118 can be decoupled from the connector 116 and activated (e.g., by depressing a plunger of the syringe) to drive blood and clot material from the pressure source 118 into the filtering device 120. Once filtered by the filtering device 120, the filtered blood can be transferred to the secondary pressure source 119 and reinfused into the patient. In some embodiments, the secondary pressure source 119 can be omitted and the separated blood can be transferred to the pressure source 118 and/or one or more other suitable pressure sources.

FIG. 2 is a perspective view of the filtering device 120 (FIG. 1) in accordance with embodiments of the present technology. In the illustrated embodiment, the filtering device 120 includes a body 222, a cover or lid 230, a filter 238, a collection component 240, and an outlet conduit 242. The body 222 can include an upper end portion or rim 224 that defines an opening 226 to an interior or chamber 228 of the body 222. The chamber 228 can be configured to receive, via the opening 226, material aspirated from a patient, including material aspirated using the catheter 106 and the pressure source 118 (FIG. 1). Referring to FIGS. 1 and 2, in some embodiments the chamber 228 has a chamber volume that is greater than a volume of the pressure source 118 (FIG. 1). For example, the chamber volume can be up to or at least two times, three times, four times, five times, etc., greater than a volume of the pressure source 118 (e.g., a barrel of a syringe). Having a chamber volume that is greater than the volume of the pressure source 118 can allow the user to empty aspirated material within the pressure source 118 after two, three, four, or more repeated aspirations into the chamber 228 without, e.g., having to empty the chamber 228 after each aspiration.

In the illustrated embodiment, the body 222 has a circular cross-section and sidewalls that are sloped/angled radially inwardly in a direction away from the rim 224. In other embodiments, the body 222 can have an oval, triangular, square, rectangular, pentagonal, and/or other suitable cross-sectional shape, and/or sidewalls that are at least substantially vertical (e.g., so that, when viewed from the side, the body 222 has a square or rectangular silhouette) or sloped/angled radially outwardly from the rim 224. In these and/or other embodiments, the body 222 can be transparent, and/or at least partially transparent, to allow a user to visualize or observe objects, material, etc. positioned within the body chamber 228, including the amount of aspirated material contained within the chamber 228 at a given time.

The lid 230 can include a first or lower end portion 232a and a second or upper end portion 232b opposite the first end portion 232a. The second end portion 232b can define an opening 234 to an interior or chamber 236 of the lid 230. The lid 230 can be configured to be releasable coupled to the body 222. For example, the first end portion 232a of the lid 230 can be coupled to the body 222 around all, or at least a portion, of the rim 224. Accordingly, when the lid 230 is coupled to the body 222, the lid chamber 236 can face toward the body chamber 228 and/or the lid opening 234 can be spaced apart from the rim 224, such as shown in FIG. 2. The lid 230 can be transparent, and/or at least partially transparent, to allow a user to visualize or observe objects, material, etc. positioned within the lid chamber 236, including the amount of aspirated material within the lid chamber 236 at a given time.

The filter 238 can be received or supported by, mounted on, positioned within, and/or otherwise coupled to the body 222. In the illustrated embodiment, for example, the filter 238 is seated within the body opening 226, e.g., radially inwardly from the rim 224. In other embodiments, the filter 238 can be positioned at least partially or fully within the body chamber 228 (e.g., below the rim 224), coupled to the lid 230, and/or have one or more other suitable positions within the device 120. The filter 238 can include one or more filter stages or layers 239 (individually identified as a first or upper filter layer 239a and a second or lower filter layer 239b, shown in cross-section in the callout view in FIG. 2) configured to separate at least blood from other material (e.g., clot material) aspirated from the patient. The filter layers 239 can be coupled to one another, can contact one another without being coupled together, and/or can be spaced apart from one another. The filter 238 can be configured to operate via gravity, e.g., to allow gravity to drive blood and/or other portions of the aspirated material through the filter 238. In these and/or other embodiments, a pressure gradient created across the filter 238 can cause blood and/or other portions of the aspirated material to move through the filter 238. For example, a vacuum pressure can be applied to the body chamber 228 to draw blood and/or other portions of the aspirated material through the filter 238 and/or a positive pressure can be applied to the lid chamber 236 to drive blood and/or other portions of the aspirated material through the filter 238.

In some embodiments, the first filter layer 239a has a larger micron rating (e.g., larger pores, lower mesh size, etc.) than the second filter layer 239b and is configured to separate larger portions of the aspirated material (e.g., clot material, thrombi, larger emboli, other coagulated blood, etc.) from other and/or smaller portions (e.g., blood, fluid, smaller emboli, etc.) of the aspirated material. For example, the first filter layer 239a can be rated for at least 100 μm, 200 μm, 300 μm, 400 μm, 500 μm, or greater, e.g., up to 1000 μm (1 mm). In these and/or other embodiments, the second filter layer 239b can have a smaller micron rating (e.g., smaller pores, higher mesh size, etc.) configured to separate blood from the aspirated material, such as any portion of the aspirated material that passes through the first filter layer 239a. For example, the second filter layer 239b can be rated for up to 10 μm, 20 μm, 30 μm, 40 μm, 50 μm, or another suitable filter rating. Accordingly, for example, the first filter layer 239a can filter a bulk amount of clot material from blood removed from a patient and then the second filter layer 239b can separate small particles from the blood such that the blood is suitable for reintroduction to the patient. In some aspects of the present technology, utilizing first and second filter layers 239a, b allows larger portions of clot material to be separated from the blood without the clot material clogging the smaller pores of the second filter layer 239b.

The collection component 240 can be positioned within the body chamber 228 below the filter 238 and configured to receive any material that passes through the filter 238. In some embodiments, the collection component 240 can include a funnel that is sloped inwardly, away from the filter 238 and/or toward the outlet conduit 242, such as shown in FIG. 2. The outlet conduit 242 can be operably coupled to the collection component 240 such that the collection component 240 is positioned between the outlet conduit 242 and the filter 238. The outlet conduit 242 can include tubing that is at least generally similar or identical in structure and/or function to the tubing sections 112a-b of FIG. 1. Accordingly, the collection component 240 can direct any materials (e.g., filtered blood) received via the filter 238 toward and/or into the outlet conduit 242, such as by the force of gravity.

In some embodiments, the second pressure source 119 (or another suitable pressure source) can be operably coupled to the outlet conduit 242, e.g., to receive material contained within the outlet conduit 242. In other embodiments, a fluid control device (e.g., at least generally similar or identical in structure and/or function to the fluid control device 114 of FIG. 1) can be operably coupled to the outlet conduit 242 and configured to prevent, or at least partially prevent, material contained within the outlet conduit 242 from leaking out of the outlet conduit 242 into the surrounding environment.

During a clot removal procedure, a user can deposit material aspirated from the patient (e.g., via the catheter 106; FIG. 1) onto the filter 238, such as by emptying aspirated material from the first pressure source 118 (FIG. 1) into the lid chamber 236 through the lid opening 234. Accordingly, the user can deposit the aspirated material into the filtering device 120 without having to operably couple the first pressure source 118 (or other vessel containing the aspirating material) to the filtering device 120, tubing, valves, ports, etc. Instead, the user can position an outlet of the first pressure source 118 (or other vessel) at, within, or through the opening 234 and actuate or otherwise empty the first pressure source 118 (or other vessel) into the lid chamber 236. This, in turn, is expected to increase the speed with which the user can empty the first pressure source 118 (or other vessel) and thereby allow the user to perform multiple or repeated aspirations in quick succession with the first pressure source 118. The aspirated material is expected to include fluids (e.g., blood) and, accordingly, the lid 230 is expected to prevent, or at least partially prevent, the fluids and/or other aspirated material from splashing or otherwise contacting the user when the user deposits the aspirated material onto the filter 238. Gravity can drive a portion of the aspirated material (e.g., blood) through the filter 238 and/or onto the collection component 240. The collection component 240 can, in turn, direct the portion of the aspirated material (e.g., filtered blood) into the outlet conduit 242, from which the user can remove the portion of the aspirated material (e.g., using the second pressure source 119) and reinfuse the removed aspirated material into the patient. In some embodiments, the user can deposit aspirated material on the filter 238 at least two, three, four, five, or more times before removing the portion of the aspirated from the outlet conduit 242-this is expected to allow multiple aspirations to be performed in quick succession, e.g., without the user having to pause to empty the filtering device 120. At any point during the clot removal procedure, the user can observe the amount of material (e.g., clot material) on the filter 238 (e.g., by looking through the at least partially transparent sidewalls of the lid 230, by removing the lid 230, and/or by looking through the lid opening 234) and/or the amount of material contained within the body chamber 228 (e.g., by looking through the at least partially transparent sidewalls of the body 222).

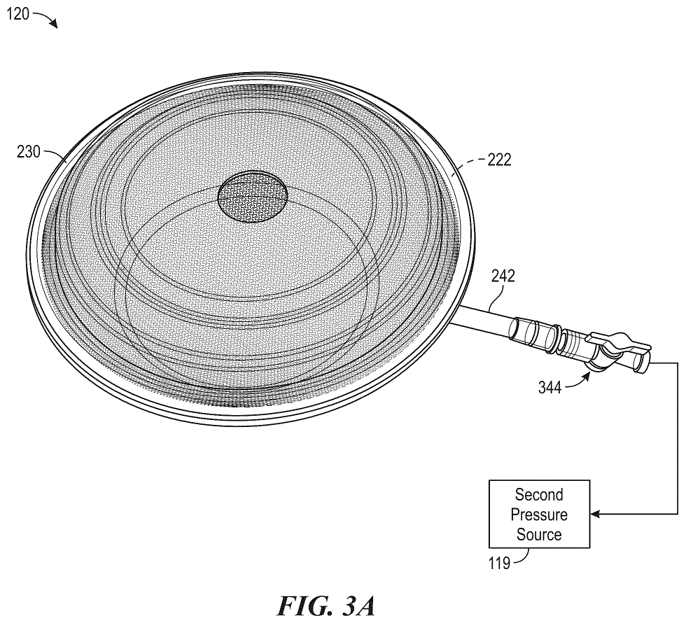

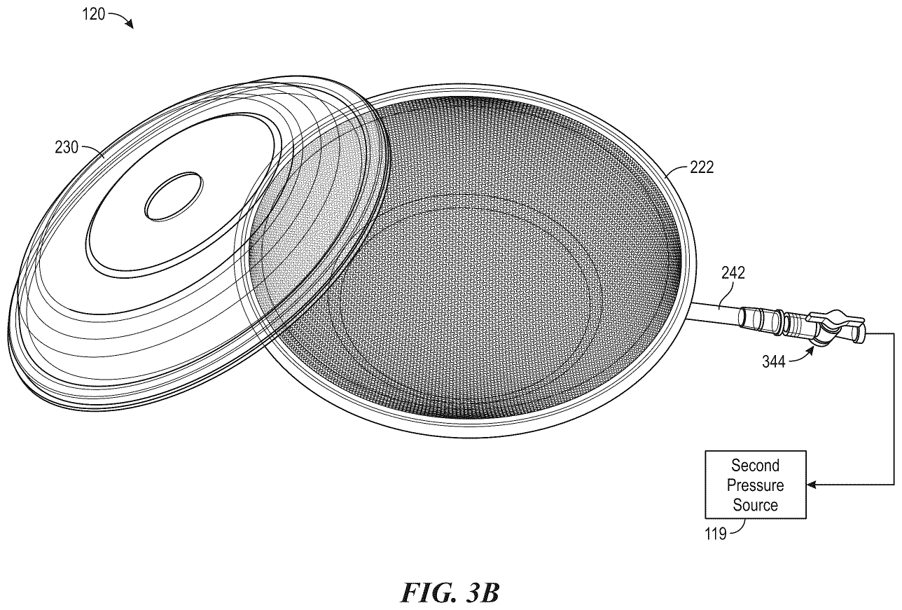

FIGS. 3A and 3B are perspective views of the device 120 of FIG. 2 in accordance with embodiments of the present technology. In FIG. 3A the lid 230 is shown coupled to the body 222. In FIG. 3B, the lid 230 is shown removed from the body 222. In FIGS. 3A and 3B, the lid 230 is shown as partially transparent for clarity. Referring to FIGS. 3A and 3B together, in some embodiments a fluid control device 344, which can be at least generally similar or identical in structure and/or function to the fluid control device 114 (FIG. 1), can be operably coupled to the outlet conduit 242 to control access to fluid within the outlet conduit 242. For example, the fluid control device 344 can be transitioned from (i) a closed position or state, in which the fluid control device 344 inhibits or even prevents material within the outlet conduit 242 from flowing through the fluid control device 344, to (ii) an open position or state, in which the fluid control device 344 allows material within the outlet conduit 242 to flow through the fluid control device 344. The second pressure source 119 (or another pressure source) can be removably coupled to the outlet conduit 242 via the fluid control device 344 and, when the fluid control device 344 is in the open position, the second pressure source 119 can be used to draw material (e.g., blood) within the outlet conduit 242 and/or otherwise contained within the body 222 through the fluid control device 344. In other embodiments, the outlet conduit 232 and the fluid control device 344 can be fluidly coupled to other containers, receptacles, etc., with or without an active pressure source.

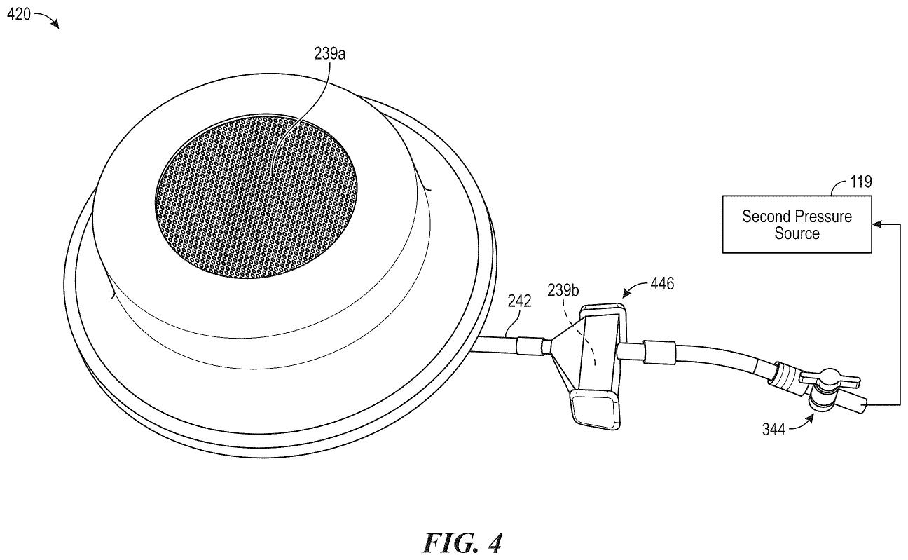

FIG. 4 is a perspective view of a filtering device 420 configured in accordance with additional embodiments of the present technology. At least some elements of the filtering device 420 can be at least generally similar or identical in structure and/or function to the filtering device 420 of FIGS. 1-3B. For example, the filtering device 420 includes the first filter layer 239a, the second filter layer 239b, the outlet conduit 242, and the fluid control device 344. In the illustrated embodiment, the filtering device 420 further includes a filter assembly 446 (e.g., a secondary filter assembly). The filter assembly 446 can contain, house, or otherwise include the second filter layer 239b and can be operably coupled to the outlet conduit 242, e.g., downstream from the first filter layer 239a and/or in series between the outlet conduit 242 and the fluid control device 344. Accordingly, the second pressure source 119 (or another pressure source) can be used to apply a pressure (e.g., vacuum pressure) to the filter assembly 446 and/or otherwise establish a pressure gradient across the second filter layer 239b to thereby cause material (e.g., blood) to flow through the second filter layer 239b. In at least some embodiments, such as when the micron rating of the second filter layer 239 is sufficiently large to create a flow resistance that inhibits, or even prevents, gravity alone from being able to sufficiently drive material (e.g., blood) across the second filter layer 239b, using the second pressure source 119 to draw material across the second filter layer 239b can overcome the flow resistance of the second filter layer 239b. Coupling the filter assembly 446 to the outlet conduit 242 can allow the outlet conduit 242 to be degassed prior to drawing blood and/or other fluids within the outlet conduit 242 through the filter assembly 446. For example, when the rating of the second filter layer 239b is configured such that the second filter layer 239 is permeable to air but, in the absence of externally-applied pressure (e.g., from the second pressure source 119), is impermeable or at least substantially impermeable to blood and/or other fluid, the fluid control device 344 can be opened to allow air and/or other gases to exit the outlet conduit 242 through the filter assembly 446 (e.g., via gravity, natural buoyancy, diffusion, etc.) while the filter assembly 446 inhibits or even prevents blood and/or other fluid within the outlet conduit 242 from passing through the filter assembly 446. After a sufficient amount of time has passed to at least partially or fully de-gas the outlet conduit 242, the user can couple the second pressure source 119 to the fluid control device 344 and draw degassed blood and/or other fluid through the filter assembly 446. Additionally, or alternatively, coupling the filter assembly 446 in series between the outlet conduit 242 and the fluid control device 344 can allow the second filter layer 239b within the filter assembly 446 to be cleaned (or the entire filter assembly 446 to be replaced) without or substantially without disassembling other portions of the filtering device 420.

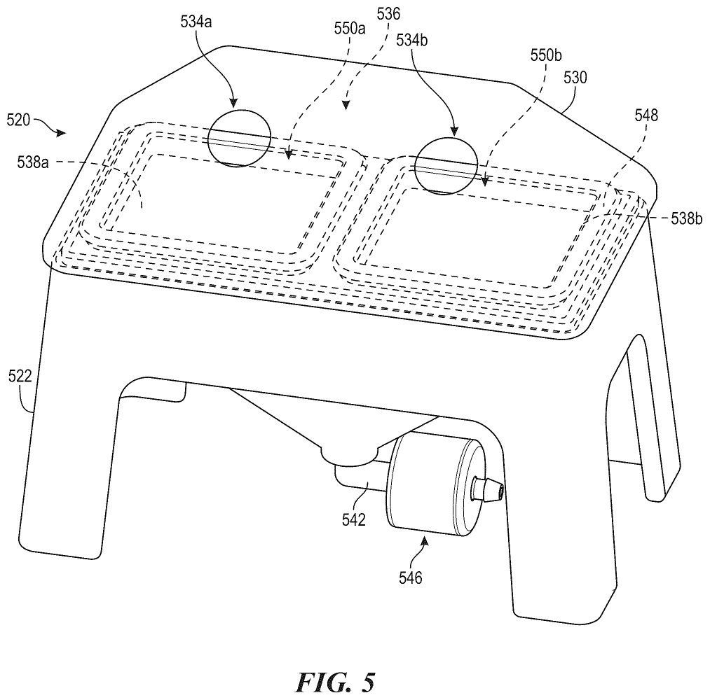

FIG. 5 is a perspective view of a filtering device 520 configured in accordance with additional embodiments of the present technology. At least some elements of the filtering device 520 can be at least generally similar or identical in structure and/or function to the filtering device 120 of FIGS. 1-3B. For example, in the illustrated embodiment the filtering device 520 includes a body 522, a lid 530 configured to be releasably coupled to the body, an outlet conduit 542, and a filter assembly 546. The body 522 can include or be configured to receive a filter support structure or plate 548 (“filter plate 548”). The filter plate 548 can define one or more openings 550 (individually identified as a first or left opening 550a and a second or right opening 550b). Each of the openings 550 can be configured to receive a corresponding filter 538 (individually identified as a first or left filter 538a and a second or right filter 538b; shown schematically with dashed-line in FIG. 5). Material (e.g., blood) that passes through the filters 538 can flow to the outlet conduit 542. In the illustrated embodiment each of the filters 538 drains to the same outlet conduit 542. In other embodiments, one or more of the filters 538 can drain to individual or respective outlet conduits. The filter assembly 546 can be fluidly coupled to the outlet conduit 542, e.g., to separate small particles from blood within the outlet conduit 542 such that the blood is suitable for reintroduction to the patient, as described previously herein.

The lid 530 can define one or more openings 534 (individually identified as a first or left opening 534a and a second or right opening 534b) to an interior or chamber 536 defined by the lid 530. Each of the lid openings 534 can be positioned above and/or otherwise aligned with a corresponding one of the filter plate openings 550 and/or the filters 538. In the illustrated embodiment, for example, the first lid opening 534a is aligned with the first filter plate opening 550a and/or the first filter 538a and the second lid opening 534b is aligned with the second filter plate opening 550b and/or the second filter 538b. Accordingly, a user can deposit aspirated material onto the first filter 538a via the first lid opening 534a and/or onto the second filter 538b via the second lid opening 534b.

In some embodiments, the user can use each of the different filters 538 and/or filter plate openings 550 to track how much material was aspirated from various locations within a patient. For example, the user can deposit first material aspirated from a first location within the patient (e.g., the left pulmonary artery, left lung, etc.) onto the first filter 538a and deposit second material aspirated from a second location within the patient (e.g., the right pulmonary artery, right lung, etc.) onto the second filter 538b. This, in turn, can allow the user to observe, track, compare, and/or otherwise quantify the amount of material aspirated from the first and second locations, which may inform further aspects of the patient's treatment.

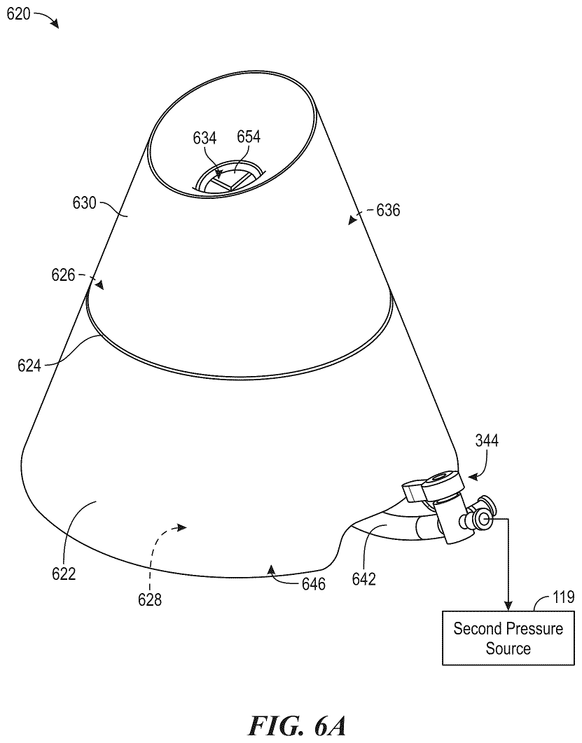

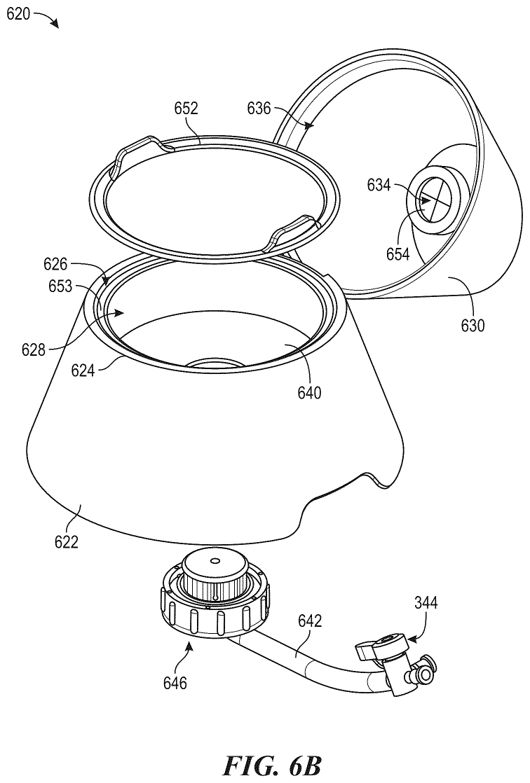

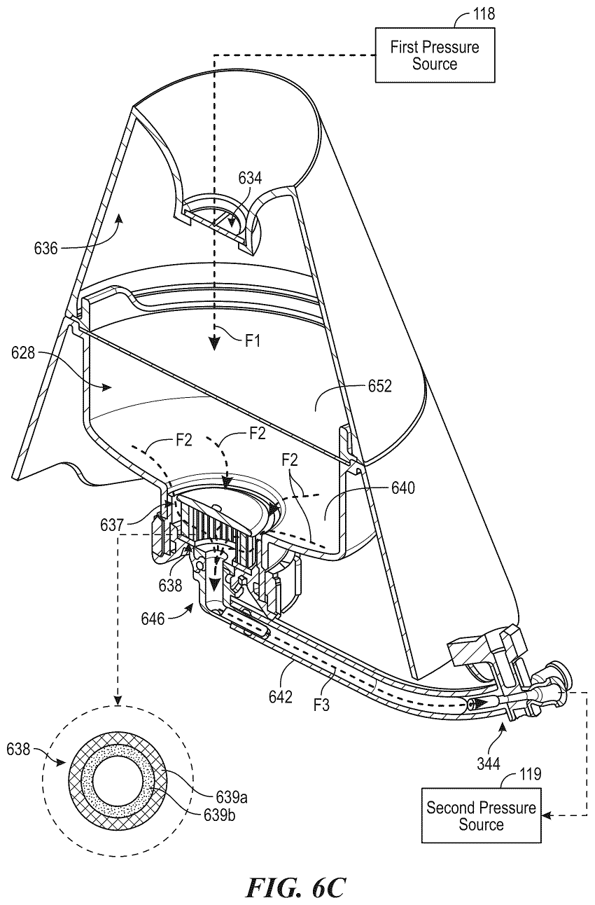

FIG. 6A is a perspective view of a filtering device 620 (e.g., filtering assembly) configured in accordance with additional embodiments of the present technology. FIG. 6B is a partially-exploded perspective view of the filtering device 620. Referring to FIGS. 6A and 6B together, at least some elements of the filtering device 620 can be at least generally similar or identical in structure and/or function to the filtering device 120 of FIGS. 1-3B, and the filtering device 620 can be utilized in the system 100 of FIG. 1 in the same or similar manner as the filtering device 120. For example, in the illustrated embodiment the filtering device 620 includes a body 622, a lid 630 configured to be selectively coupled to the body 622 (via, e.g., a hinge or other coupling mechanism), an outlet conduit 642, and a filter assembly 646 (FIG. 6B; obscured in FIG. 6A). The body 622 can include an upper end portion or rim 624 that defines an opening 626 to an interior or chamber 628 of the body 622. The chamber 628 can be configured to receive, via the opening 626, material aspirated from a patient, including material aspirated using the catheter 106 and the pressure source 118 (FIG. 1), such as blood and clot material. The body 622 can be transparent, and/or at least partially transparent, to allow a user to visualize or observe objects, material, etc. positioned within the body chamber 628, including the amount of aspirated material within the body chamber 628 at a given time. In some embodiments, a collection component 640 (FIG. 6B) can be positioned within the body chamber 628 in, e.g., a lower portion thereof. The collection component 640 can be sloped inwardly and/or otherwise configured to direct fluid and/or other material within the body chamber 628 toward the filter assembly 646.

In some embodiments, the body 622 can be configured to receive a filter tray 652, e.g., in an upper portion thereof. In the illustrated embodiment, for example, the body 622 is configured to receive and support the filter tray 652 on a lip or shelf 653 (FIG. 6B; obscured in FIG. 6A) radially inward from the rim 624 and/or at least partially within the opening 626 to the body chamber 628. For example, the filter tray 652 can rest on the lip 653 and be removable therefrom, e.g., for cleaning. The filter tray 652 can be positioned at least partially between the lid 630 and the body 622 so that any aspirated material that enters the filtering device 620 via, e.g., the lid 630, must pass through the filter tray 652 before entering the body chamber 628. The filter tray 652 can have a micron rating equal to or greater than one or more filters in the filter assembly 646. In at least some embodiments, for example, the filter tray 652 has a micron rating of at least 500 μm, 600 μm, 700 μm, 800 μm, 900 μm, 1000 μm, 1100 μm or greater, e.g., up to 2000 μm or 3000 μm. Accordingly, the filter tray 652 can be configured to inhibit, or even prevent, larger portions of the aspirated material (e.g., clot material, thrombi, larger emboli, other coagulated blood, etc.) received via the pressure source 118 from entering into the body chamber 628, e.g., to allow the filter assembly 646 to have a relatively finer micron rating. As such, the filter tray 652 is expected to inhibit, or even prevent, larger portions of the aspirated material from clogging the filter assembly 646.

The lid 630 can define an opening 634 to an interior or chamber 636 of the lid 630. When the lid 630 is coupled to the body 622, the lid chamber 636 can face toward the body chamber 628 and/or the lid opening 634 can be spaced apart from the rim 624, such as shown in FIGS. 6A and 6B. In some embodiments, the lid 630 and/or the body 622 include a coupling mechanism (e.g., press-fit coupling, threading, coupling flanges, latches, hinges, etc.) configured to keep the lid 630 securely coupled to the body 622, e.g., while the filtering device 620 is in use. The lid 630 can be transparent, and/or at least partially transparent, to allow a user to visualize or observe objects or material positioned within the lid chamber 636, such as the amount of aspirated material within the lid chamber 636 at a given time. In some embodiments, the lid opening 634 can include a valve 654 configured to control access to the lid chamber 636. In the illustrated embodiment, for example, the valve 654 includes a cross-slit valve configured to at least partially prevent access to the lid chamber 636. The flaps or other sealing members of the cross-slit valve can be configured such that, when a user wants to deposit aspirated material within the lid chamber 636, the user can easily defeat the cross-slit valve by, e.g., inserting a tip of the first pressure source 118 (FIG. 1) through the flaps or other sealing members. The valve 654 can inhibit or even prevent backsplash of the aspirated material and contain the aspirated material within the filtering device 620. In at least some embodiments, the user can deposit aspirated material within the lid chamber 636 without, e.g., needing to couple or otherwise operably engage the first pressure source 118 with the filtering device 620. That is, the valve 654 can be omitted.

The filter assembly 646 can be positioned downstream (e.g., vertically beneath or at an elevation below) the body chamber 628 and can be configured to receive material therefrom. The outlet conduit 642 can be coupled to the filter assembly 646 and configured to receive material that passes therethrough. The second pressure source 119 (FIG. 1) can be operably coupled to the outlet conduit 642 via, e.g., the fluid control device 344 to allow the second pressure source 119 to receive material from the outlet conduit 642 and/or draw material through the filter assembly 646, as described in greater detail below.

FIG. 6C is a perspective cross-sectional view of the filtering device 620 of FIGS. 6A in accordance with embodiments of the present technology. In the illustrated embodiment, the filter assembly 646 includes a filter 638 positioned to receive material from the body chamber 628. In the illustrated embodiment, the filter 638 is positioned within a filter chamber 637 (e.g., a receiving portion) located at least partially below the collection component 640. The collection component 640 can be sloped (e.g., downwardly) toward the filter 638 so that material on the collection component 640 can move/flow (by, e.g., gravity) toward and into the filter chamber 637, and into and through the filter 638 therein.

The filter 638 can be annular and configured to direct fluid flow radially inwardly thereacross. The filter 638 can, additionally or alternatively, be pleated to, e.g., increase the surface area available for filtering and/or otherwise improve the performance of the filter 638. The filter 638 can include one or more filter stages or layers 639 (individually identified as a first or radially outer filter layer 639a and a second or radially inner filter layer 639b, shown in detail in an enlarged cross-sectional top view in FIG. 6C) configured to separate at least blood from other material (e.g., clot material) aspirated from the patient. The filter layers 639 can be coupled to one another, can contact one another without being coupled together, and/or can be spaced apart from one another. The filter 638 can be configured to operate via gravity, e.g., to allow gravity to drive blood and/or other portions of the aspirated material through the filter 638. In these and/or other embodiments, a pressure gradient created across the filter 638 can cause blood and/or other portions of the aspirated material to move through the filter 638. For example, vacuum pressure from, e.g., the second pressure source 119 can be applied to the filter 638, the filter chamber 637, and/or the body chamber 628 to draw blood and/or other portions of the aspirated material through the filter 638 and/or a positive pressure can be applied to the lid chamber 636 to drive blood and/or other portions of the aspirated material through the filter 638.

In some embodiments, the first filter layer 639a has a larger micron rating (e.g., larger pores, lower mesh size, etc.) than the second filter layer 639b and is configured to separate larger portions of the aspirated material (e.g., clot material, thrombi, larger emboli, other coagulated blood, etc.) from other and/or smaller portions (e.g., blood, fluid, smaller emboli, etc.) of the aspirated material. For example, the first filter layer 639a can be rated for at least 100 μm, 200 μm, 300 μm, 400 μm, 500 μm, or greater, e.g., up to 1000 μm (1 mm). In these and/or other embodiments, the second filter layer 639b can have a smaller micron rating (e.g., smaller pores, higher mesh size, etc.) configured to separate blood from the aspirated material, such as any portion of the aspirated material that passes through the first filter layer 639a. For example, the second filter layer 639b can be rated for up to 10 μm, 20 μm, 30 μm, 40 μm, 50 μm, or another suitable filter rating. Accordingly, for example, the first filter layer 639a can filter a bulk amount of clot material from blood removed from a patient and then the second filter layer 639b can separate small particles from the blood such that the blood is suitable for reintroduction to the patient. In some aspects of the present technology, utilizing first and second filter layers 639a, b allows larger portions of clot material to be separated from the blood without the clot material clogging the smaller pores of the second filter layer 639b.

Referring to FIGS. 6A-6C, during a clot removal procedure, a user can deposit material aspirated from the patient (e.g., via the catheter 106; FIG. 1) onto the filter tray 652, such as by emptying aspirated material from the first pressure source 118 (FIG. 1), and/or one or more other vessels containing the aspirated material, into the lid chamber 636 through the lid opening 634, as shown by arrow F1. Accordingly, the user can deposit the aspirated material into the filtering device 620 without having to operably couple the first pressure source 118 to the filtering device 620, tubing, valves, ports, etc. Instead, the user can position an outlet of the first pressure source 118 at, within, or through the opening 634 and actuate or otherwise empty the first pressure source 118 into the lid chamber 636. This, in turn, is expected to increase the speed with which the user can empty the first pressure source 118 and thereby allow the user to perform multiple or repeated aspirations in quick succession with the first pressure source 118. The aspirated material is expected to include fluids (e.g., blood) and, accordingly, the lid 630 is expected to prevent, or at least partially prevent, the fluids and/or other aspirated material from splashing or otherwise contacting the user when the user deposits the aspirated material onto the filter tray 652. Gravity can drive a portion of the aspirated material (e.g., blood) through the filter tray 652 and/or onto the collection component 640. The collection component 640 can, in turn, direct the portion of the aspirated material (e.g., filtered blood) toward, into, and/or through the filter assembly 646, as shown by arrows F2. In some embodiments, the user can use the second pressure source 119 to draw material (e.g., blood) within the body chamber 628 and/or the filter chamber 637 through the filter 638 into the filter assembly 646. In these and/or other embodiments, gravity can drive a portion of the material (e.g., blood) within the body chamber 628 through the filter 638 into the filter assembly 646. The material (e.g., blood) that passes through the filters 638 can flow to the outlet conduit 642, as shown by arrow F3. The user can remove the material within the outlet conduit 642 (e.g., using the second pressure source 119) and reinfuse it into the patient. In at least some embodiments, for example, the user can couple the second pressure source 119 to fluid control device 344 and open and/or close the fluid control device 344 as needed to remove fluid from the outlet conduit using the second pressure source 119.

Put differently, the filtering device 620 can receive blood and clot material into lid chamber 636 through the valve 654 from the first pressure source 118 (FIG. 1). The blood and clot material can move (e.g., flow) downward toward/onto/through the filter tray 652 via gravity. The filter tray 652 can inhibit or even prevent larger portions of the aspirated material (e.g., larger portions of the clot material, coagulated blood) from flowing therethrough while permitting smaller portions of the aspirated material (e.g., blood, smaller portions of the clot material) to move therethrough downward toward/onto the collection component 640. Accordingly, the filter tray 652 provides a first filter stage that filters out large portions of the clot material. After the filter tray 652 has collected a sufficient quantity of the large portions of the clot material, the user can remove the filter tray 652, e.g., for cleaning, and the user can remove all or at least a portion of the clot material collected by the filter tray 652. The collection component 640 can direct the first-stage filtered material toward the filter 638 positioned within the filter chamber 637. The second pressure source 119 (FIG. 1) can be coupled to the outlet tube 642 via the fluid control device 344 and, with the fluid control device 344 in an open position, the second pressure source 119 can be used to generate negative pressure to draw the first-stage filtered material through the filter 638, through the outlet conduit 642, and through the fluid control device 344 into the second pressure source 119. The filter 638 can provide a second filtering stage that filters out smaller portions of clot material while permitting blood to pass therethrough. Additionally or alternatively, the first-stage filtered material can flow through the filter 638 at least partially due to gravity. Accordingly, the filtering device 620 is configured to receive aspirated blood and clot material from the first pressure source 118 (and/or another source of aspirated material), filter the clot material from the blood, and permit the filtered blood to pass to the second pressure source 119 (and/or another container). In some aspects of the present technology, the filter tray 652 can filter out large portions of the clot material that may otherwise clog or interfere with the operation of the filter 638.

The body chamber 628 is configured to receive the aspirated blood and clot material and, in some embodiments, can have a volume greater or significantly greater than the volume of the first pressure source 118. For example, the body chamber 628 can have a volume equal to or greater than about 100 cc, about 200 cc, about 300 cc, about 400 cc, about 500 cc, about 600 cc, about 1000 cc, and/or the like. Accordingly, the body chamber 628 can be sized to store/hold material aspirated multiple times via the first pressure source 118 and/or another pressure source or aspiration container. Therefore, in some embodiments, a user (e.g., a surgeon and/or healthcare team member) can deposit aspirated material into the filtering device 620 at least two, three, four, five, or more times before removing the portion of the aspirated from the outlet conduit 642—this is expected to allow multiple aspirations to be performed in quick succession, e.g., without the user having to pause and empty the filtering device 620 each time aspirated material is deposited therein. At any point during the clot removal procedure, the user can observe the amount of material (e.g., clot material) on the filter tray 652 (e.g., by looking through the at least partially transparent sidewalls of the lid 630, by removing the lid 630, and/or by looking through the lid opening 634) and/or the amount of material contained within the body chamber 628 (e.g., by looking through the at least partially transparent sidewalls of the body 622).

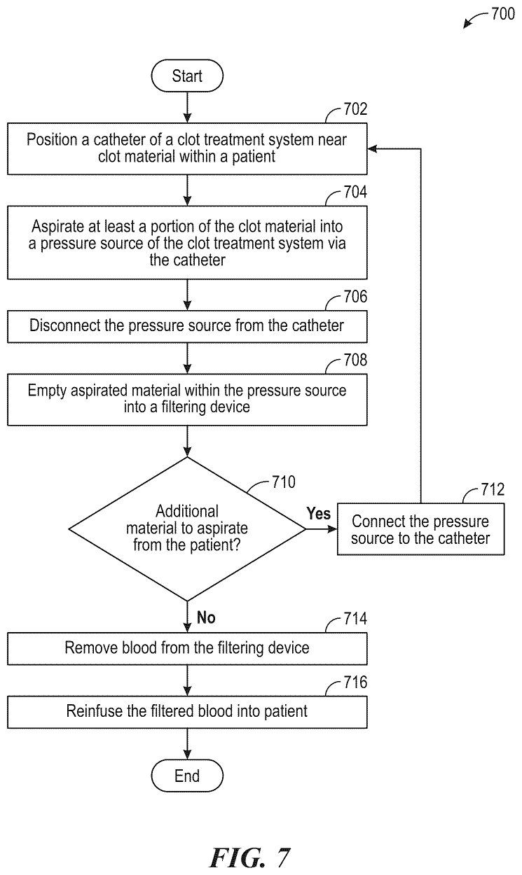

FIG. 7 is a flow diagram of a method 700 for removing clot material from a patient. The method 700 is illustrated as a series of steps, process portions, or blocks 702-716. One or more of the blocks 702-716 of the method 700 can be performed using one or more of the devices and/or systems described herein. For example, several blocks of the method 700 are described with reference to the filtering device 120 (FIGS. 1-3B). However, a person of ordinary skill in the art will appreciate that the filtering device 420 (FIG. 4), the filtering device 520 (FIG. 5), and/or the filtering device 620 (FIGS. 6A-6C) can also be used to perform one or more blocks of the method 700.

At block 702, the method 700 can include positioning a catheter of a clot treatment system near clot material within a patient. Positioning the catheter can include positioning the catheter 106 of the system 100 near clot material within a blood vessel of the patient, as described previously herein and at least with reference to FIG. 1.

At block 704, the method 700 can include aspirating at least a portion of the clot material into a pressure source of the clot treatment system via the catheter. Aspirating the clot material into the pressure source can include apply a vacuum stored within the pressure source 118 to the catheter 106, as described previously herein at least with reference to FIG. 1.

At block 706, the method 700 can include disconnecting the pressure source from the catheter. Disconnecting the pressure source can include disconnecting the pressure source 118 from the valve 102 or the connector 116, as described previously herein at least with reference to FIG. 1.

At block 708, the method 700 can include emptying aspirated material within the pressure source into a filtering device. The filtering device can be configured to filter blood from the other aspirated material that it receives. Emptying aspirated material into the filtering device can include emptying aspirated material into one or more of the filtering devices 120, 420, 520, 620. For example, emptying the aspirated material into the filtering device can include positioning the pressure source 118 near, within, or through the lid opening 234 of the filtering device 120 and emptying aspirated material contained within the pressure source 118 into the lid chamber 236 via the lid opening 234. When the pressure source 118 includes a syringe, emptying the aspirated material into the lid chamber 236 can include pressing a handle of the syringe to empty aspirated material contained within a barrel of the syringe. Emptying aspirated material into the lid chamber 236 can include depositing the aspirated material onto a filter contained within the filtering device, such as the filter 238 (FIG. 2) of the filtering device 120 or the filter tray 652 (FIGS. 6B and 6C).

At block 710, the method 700 can include determining whether there is additional material to aspirate from the patient. If there is additional material to aspirated from the patient (block 710, YES), the method 700 can include block 712. If not (block 710, NO), the method 700 can include block 714.

At block 712, the method 700 can include connecting (e.g., reconnecting) the pressure source to the catheter and/or repeating one or more of the blocks 702-710 of the method 700. For example, the user can connect the pressure source 118 to the catheter 106 can aspirate additional material from the patient, as described previously with reference to one or more of blocks 702-710.

At block 714, the method 700 can include removing blood from the filtering device. Removing blood from the filtering device can include using a syringe or other pressure source, such as the second pressure source 119, to remove blood from the filtering device, as described previously herein and at least with reference to FIGS. 1 and 2. In some embodiments, filtered blood can drain into a container connected to an outlet conduit of the filtering device without being actively aspirated from the filtering device. In some embodiments, removing blood from the filtering device can include removing blood from the filtering device via an outlet conduit and/or a fluid control device, such as the outlet conduit 242 (FIG. 2) and/or the fluid control device 344 (FIGS. 3A and 3B). Additionally, or alternatively, removing blood from the filtering device can include drawing the blood through a filter to separate the blood from one or more other portions of the aspirated material, such as by drawing the blood through the second filter layer 239b of the filter assembly 446 (FIG. 4) and/or the filter 638 (FIG. 6C).

At block 716, the method 700 can include reinfusing the filtered blood into the patient. Reinfusing the filter blood into the patient can include using the second pressure source 119 (or another pressure source, container, etc.) to reinfuse the filtered blood, as described previously herein at least with reference to FIG. 1.

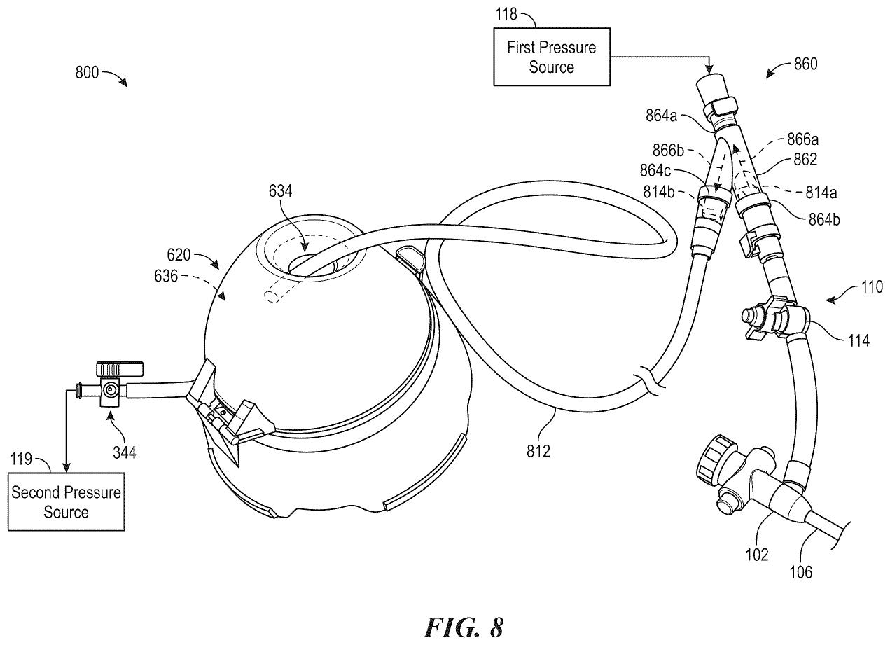

FIG. 8 is a partially schematic perspective view of a clot treatment system 800 (“the system 800”) configured in accordance with additional embodiments of the present technology. The system 800 can also be referred to as an aspiration assembly, a vascular access system, a clot removal system, a thrombectomy system, and/or the like. At least some aspects of the system 800 can be at least generally similar or identical in structure and/or function to one or more aspects of the system 100 of FIG. 1. In the illustrated embodiment, for example, the system 800 includes the valve 102, the catheter 106, the flow control device 114, the tubing assembly 110, the first pressure source 118, and the second pressure source 119. In some embodiments, the first pressure source 118 can include features generally similar in structure and/or function, or identical in structure and/or function, to one or more of the syringes (e.g., an automatic locking an unlocking vacuum syringe) and/or other pressure sources described in detail in U.S. patent application Ser. No. 18/949,343, filed Nov. 15, 2024, and titled “AUTOMATIC LOCKING AND UNLOCKING VACUUM SYRINGES, AND ASSOCIATED SYSTEMS AND METHODS,” which is incorporated by reference herein in its entirety. The system 800 can also include one or more of the filtering devices described herein, e.g., with reference to one or more of FIGS. 1-7. In the illustrated embodiment, for example, the system 800 includes the filtering device 620 of FIGS. 6A-6C.

The system 800 can further include a connector 860 positioned to fluidly couple (i) the catheter 106 to the first pressure source 118 via the tubing assembly 110 and (ii) the first pressure source 118 to the filter device 620 via tubing 812. The connector 860 can include a body 862 that defines one or more openings or ports 864 (individually identified as a first port 864a, a second port 864b, and a third port 864c) and one or more flow paths 866 (individually identified as a first flow path 866a and a second flow path 866b). The body 862 can be branched, bifurcated, or y-shaped. Each of the ports 864 can be located at or proximate to a respective end or terminus of the body 862 and can be configured to be coupled (e.g., fluidly coupled) to one or more other elements in the system 800. In the illustrated embodiment, the first port 864a is coupled to the first pressure source 118, the second port 864b is coupled to the catheter 106 (via, e.g., the valve 102 and the tubing assembly 110), and the third port 864c is coupled to the tubing 812. At least a portion of the tubing 812 is configured to be positioned through the opening 634 and/or otherwise received within the filtering device 620 (e.g., within the lid chamber 636 of the filtering device 620).

Each of the flow paths 866 can be defined at least partially by a corresponding lumen or space within the body 862 extending between individual ones of the ports 864. In the illustrated embodiment, the first flow path 866a extends between the first and second ports 864a, 864b and the second flow path 866b extends between and/or fluidly couples the first and third ports 864a, 864c. Accordingly, the first flow path 866a can direct fluid and/or other material aspirated from a patient via the catheter 106 toward and/or into the first pressure source 118. The second flow path 866b can direct the fluid and/or other material from the first pressure source 118 toward and/or into the filtering device 620 (via, e.g., the tubing 812). As described in further detail below, the connector 860 can be configured to fluidly couple the catheter 106 to the filtering device 620 in series (via, e.g., the ports 864 and/or the flow paths 866) to allow a user/operator to repeatedly aspirate material using the catheter 106 and transfer the aspirated material to the filtering device 620 without needing to disconnect and/or reconnect the pressure source 118 from other elements of the system (e.g., as described with reference to blocks 706 and 712 in FIG. 7) before doing so. In other embodiments, one or more of the ports 864 can be connected to additional and/or other elements of the system 800.

In some embodiments, the connector 860 includes one or more fluid control devices 814 (individually identified as a first fluid control device 814a and a second fluid control device 814b) positioned and/or otherwise configured to control flow along the first flow path 866a and/or the second flow path 866b. The first fluid control device 814a can be positioned at least partially within and/or otherwise operably coupled to the second port 864b and can be configured to (i) permit fluid flow therethrough, e.g., through the second port 864b into the connector 860 along the first flow path 866a to the first pressure source 118, and (ii) inhibit or even prevent fluid flow therethrough, e.g., through the second port 864b along the first flow path 866a from the first pressure source 118 toward the tubing assembly 110 and the catheter 106. Similarly, the second fluid control device 814b can be positioned at least partially within and/or otherwise operably coupled to the third port 864c and can be configured to (i) permit fluid flow therethrough, e.g., through the third port 864c into the tubing 812 along the second flow path 866b and (ii) inhibit or even prevent fluid flow therethrough, e.g., through the third port 864c along the second flow path 866b from the tubing 812 to the first pressure source 118. The first and second fluid control devices 814a, 814b can be one-way or check valves. In other embodiments, one or more of the fluid control devices 814 can include a ball valve, a gate valve, and/or one or more other fluid control devices (including, e.g., the fluid control device 114 of FIG. 1) that are configured to be externally operable by a user.

In at least some embodiments, the one or more of the fluid control devices 814 can be operated at least partially in response to a pressure generated by the pressure source 118 and/or otherwise applied to the first port 864a. For example, when a first (e.g., vacuum) pressure is generated by the pressure source 118 and/or otherwise applied to the first port 864a, such as when aspirating clot material within a patient, the first fluid control device 814a can be open and the second fluid control device 814b can be closed so as to direct fluid and/or other material along the first flow path 866a, toward and/or through the first port 864a and/or into the first pressure source 118. In contrast, when a second (e.g., positive) pressure is generated by the pressure source 118 and/or otherwise applied to the first port 864a, such as when aspirated material is expelled from the pressure source 118, the first fluid control device 814a can be closed and the second fluid control device 814b can be open so as to direct the aspirated material along the second flow path 866b, outwardly from the first pressure source 118, through the third port 864c, into the tubing 812, and into the filtering device 620. This selective opening and closing of the first and second fluid control devices 814a, 814b can occur automatically if, e.g., the first and second fluid control devices 814a, 814b include respective one-way or check valves positioned at opposite orientations relative to their respective ports 864b, 864c. In other embodiments, the first fluid control device 814a and/or the second fluid control device 814b can be opened and closed manually to direct fluid flow along the first flow path 866a and/or the second flow path 866b.

During a clot removal procedure, the connector 860 can be configured to allow a user/operator to repeatedly aspirate material from a patient into a syringe or other pressure source and transfer that aspirated material to a filtering device, without the user/operator needing to uncouple the syringe/other pressure source from the catheter or other device used to aspirate that material before transferring the aspirated material to the filtering device. For example, during a clot removal procedure, the user can close the fluid control device 114 and generate and/or store a vacuum pressure within the first pressure source 118. This vacuum pressure can open the first fluid control device 814a and close the second fluid control device 814b. The user can apply the vacuum pressure to the catheter 106 (via, e.g., the first port 864a) and thereby aspirate material from the patient by opening the fluid control device 114. The aspirated material can flow through the tubing assembly 110, through the first fluid control device 814a, along at least a portion of the first flow path 866a, and/or into the pressure source 118. The user can then generate a positive pressure within the first pressure source 118 by, e.g., depressing a plunger of a syringe, to expel the aspirated material. The positive pressure can close the first fluid control device 814a and open the second fluid control device 814b to direct the aspirated material along at least a portion of the second flow path 866b, out from the connector 860 via the third port 864c, through at least a portion of the tubing 812, and/or into the filtering device 620. Because the fluid control devices 814 are configured to direct fluid flow through the connector 860 along the various flow paths 816 in response to the pressure generated by the pressure source 118, the user/operator can repeat the above-noted process as needed to both aspirate additional clot material from the patient and/or discharge/expel that additional clot material into the filtering device 620 without needing to uncouple the first pressure source 118 from the connector 860 to, e.g., empty aspirated material from within the first pressure source 118. Any clot material received within the filtering device 620 can be filtered to, e.g., remove blood (as described previously with reference to at least FIGS. 6A-6C) and at least a portion of the blood or other filtered material can be removed by the second pressure source 119 (via, e.g., the fluid control device 344).

In some embodiments, rather than closing the fluid control device 114 before activating the first pressure source 118 to generate vacuum pressure, the fluid control device 114 can be opened before the first pressure source 118 generates the vacuum pressure. The vacuum pressure is therefore directly applied to the catheter 106 (e.g., as it is generated) to aspirate material through the tubing assembly 110, through the first fluid control device 814a, along at least a portion of the first flow path 866a, and/or into the first pressure source 118. The user can then generate positive pressure with the first pressure source 118 to drive the aspirated material along at least a portion of the second flow path 866b, out from the connector 860 via the third port 864c, through at least a portion of the tubing 812, and/or into the filtering device 620.

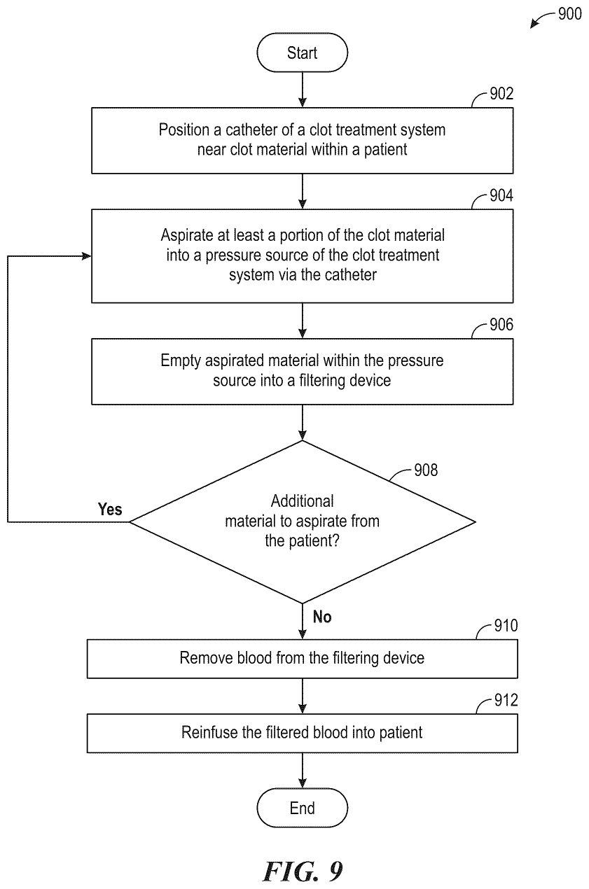

FIG. 9 is a flow diagram of a method 900 for removing clot material from a patient. The method 900 is illustrated as a series of steps, process portions, or blocks 902-912. One or more of the blocks 902-912 of the method 900 can be performed using one or more of the devices and/or systems described herein. For example, several blocks of the method 900 are described with reference to the system 800 (FIG. 8). However, a person of ordinary skill in the art will appreciate that the system 100 and/or any of the filtering devices described herein can also be used to perform one or more blocks of the method 900.

At block 902, the method 900 can include positioning a catheter of a clot treatment system near clot material within a patient. Positioning the catheter can include positioning the catheter 106 of the system 800 near clot material within a blood vessel of the patient, as described previously herein and at least with reference to FIG. 1.

At block 904, the method 900 can include aspirating at least a portion of the clot material into a pressure source of the clot treatment system via the catheter. Aspirating the clot material into the pressure source can include applying a vacuum stored within the first pressure source 118 to the catheter 106 (e.g., a vacuum generated in the first pressure source 118 with the fluid control device 114 closed), or simultaneously generating a vacuum with the first pressure source 118 and applying the vacuum to the catheter 106 (e.g., a vacuum generated with the first pressure source 118 with the fluid control device 114 opened), as described previously herein at least with reference to FIG. 1. In some embodiments, aspirating the clot material can include opening a first fluid control device (e.g., the first fluid control device 814a of FIG. 8) of a connector (e.g., connector 860) and closing a second fluid control device (e.g., the second fluid control device 814 of FIG. 8) of the connector to direct the aspirated clot material along a first flow path (e.g., the first flow path 866a) of the connector and into the pressure source.

At block 906, the method 900 can include emptying aspirated material within the pressure source into a filtering device. The filtering device can include one or more of the filtering devices 120, 420, 520, 620 described herein. Emptying the aspirated material into the filtering device can include closing a first fluid control device (e.g., the first fluid control device 814a of FIG. 8) of a connector (e.g., connector 860) and opening a second fluid control device (e.g., the second fluid control device 814 of FIG. 8) of the connector to direct the aspirated clot material along a second flow path (e.g., the second flow path 866b) of the connector such that the pressure source 118 discharges or expels all, or at least a portion, of the aspirated material along the second flow path, toward and/or into the filtering device (via, e.g., the tubing 812 of FIG. 8). When the pressure source 118 includes a syringe, emptying the aspirated material into the filtering device can include actuating a plunger of the syringe to empty aspirated material contained within a barrel of the syringe into the filtering device. In some embodiments, emptying the aspirated material into the filtering device can include emptying the aspirated material into a lid chamber (e.g., the lid chamber 636) of the filtering device (e.g., the filtering device 620 of FIGS. 6A-6C) via a lid opening (e.g., the lid opening 634 of FIGS. 6A-6C) of the filtering device. Emptying aspirated material into the lid chamber can include depositing the aspirated material onto a filter (e.g., the filter tray 652 of FIGS. 6B and 6C) contained within the filtering device.

Block 906 and/or the method 900 can omit certain steps associated with disconnecting and/or reconnecting the pressure source from or to other aspects of the clot treatment system (e.g., blocks 706 and 712 of FIG. 7) before, while, and/or after emptying the aspirated material into the filtering device. For example, emptying the aspirated material into the filtering device in block 906 can include emptying the aspirated material without disconnecting the pressure source (block 904) from the catheter (block 902) and/or connecting the pressure source to the filtering device (block 908). This is described previously herein with reference to at least FIGS. 6A-6C and 8 and is expected to improve the speed and/or efficiency with which the user can repeatedly aspirated material and transfer that aspirated material to the filtering device.

At block 908, the method 900 can include determining whether there is additional material to aspirate from the patient. If there is additional material to aspirated from the patient (block 908, YES), the method 900 can return to block 904. If not (block 908, NO), the method 900 can include block 910. When returning to block 904, the user need not connect the pressure source (block 904) to the catheter (block 902) and/or disconnect the pressure source from the filtering device (block 908) for the same reasons set forth above with reference to block 906.

At block 910, the method 900 can include removing blood from the filtering device. Removing blood from the filtering device can include using a syringe or other pressure source, such as the second pressure source 119, to remove blood from the filtering device, as described previously herein and at least with reference to FIGS. 1, 2, and 8. In some embodiments, filtered blood can drain into a container connected to an outlet conduit of the filtering device without being actively aspirated from the filtering device. In some embodiments, removing blood from the filtering device can include removing blood from the filtering device via an outlet conduit and/or a fluid control device, such as the fluid control device 344 (FIG. 8). Additionally, or alternatively, removing blood from the filtering device can include drawing the blood through a filter to separate the blood from one or more other portions of the aspirated material, such as by drawing the blood through the second filter layer 239b of the filter assembly 446 (FIG. 4) and/or the filter 638 (FIG. 6C).

At block 912, the method 900 can include reinfusing the filtered blood into the patient. Reinfusing the filter blood into the patient can include using the second pressure source 119 (or another pressure source, container, etc.) to reinfuse the filtered blood, as described previously herein at least with reference to FIG. 1.

The following examples are illustrative of several embodiments of the present technology:
1. A filtering device, comprising:
a body defining a body chamber;
a lid coupled to the body and defining a lid chamber configured to receive material aspirated from a patient;
a filter tray configured to be positioned at least partially between the body chamber and the lid chamber and to permit a first portion of the material to pass from the lid chamber into the body chamber;
a filter assembly configured to receive the first portion of the material from the body chamber and permit a second portion of the material to pass therethrough; and
an outlet conduit fluidly coupled to the filter assembly and configured to allow the second portion of the material to be removed therefrom.
2. The filtering device of example 1 wherein the material aspirated from the patient includes clot material from within a patient and wherein the first and second portions of the material include blood.
3. The filtering device of example 1 or example 2 wherein the filter assembly includes an annular filter with one or more filter stages and configured to direct the second portion of the material radially inwardly across the one or more filter stages.
4. The filtering device of example 3 wherein the one or more filter stages includes a first filter stage having a first rating and a second filter stage having a second rating different than the first rating.
5. The filtering device of example 4 wherein the first rating is at least 100 μm and wherein the second rating is up to 50 μm.
6. The filtering device of any one of examples 1-5 wherein the lid includes a valve configured to control access to the lid chamber.
7. The filtering device of example 6 wherein the valve includes a cross-slit valve.
8. The filtering device of example 6 or example 7 wherein the valve is configured to receive the material aspirated from the patient via a pressure source and without being operably coupled to the pressure source.
9. The filtering device of any one of examples 6-8 wherein the valve is configured to receive the material aspirated from the patient via a syringe and without being operably coupled to the syringe.
10. The filtering device of any one of examples 1-9, further comprising a fluid control device operably coupled to the outlet conduit and configured to be transitioned from (i) a closed state, in which the fluid control device inhibits or prevents the second portion of the material within the outlet conduit from flowing through the fluid control device, to (ii) an open state, in which the fluid control device allows the second portion of the material within the outlet conduit to flow through the fluid control device.
11. The filtering device of any one of examples 1-9 wherein the body defines an opening to the body chamber and wherein the filter tray is positioned at least partially within the opening.
12. A method of treating clot material within a patient, the method comprising:
repeatedly aspirating clot material into a pressure source of a clot treatment system via a catheter of the clot treatment system;
after one or more of the aspirations, emptying at least a portion of the aspirated clot material contained within the pressure source onto a filter of a filtering device; and
causing the filter to separate blood from the portion of the aspirated clot material.
13. The method of example 12 wherein causing the filter to separate the blood includes allowing gravity to drive the blood through the filter.
14. The method of example 12 or example 13 wherein causing the filter to separate the blood includes creating a pressure gradient across the filter to drive the blood through the filter.
15. The method of example 14 wherein creating the pressure gradient includes applying a vacuum pressure to a downstream side of the filter.
16. The method of example 14 or example 15 wherein creating the pressure gradient includes applying a positive pressure to an upstream side of the filter.
17. The method of any one of examples 12-16, further comprising:
removing the filtered blood from the filtering device, and
reinfusing the filtered blood into a patient.
18. The method of example 17 wherein:
the filtering device includes an outlet conduit configured to contain the filtered blood and a fluid control device configured to control access to the filtered blood within the outlet conduit,
removing the filtered blood includes—
operably coupling a syringe or other vessel to the fluid control device, and
receiving the filtered blood within the syringe or other vessel; and reinfusing the filtered blood includes reinfusing the filtered blood using the syringe or other vessel.
19. The method of any one of examples 12-18 wherein the filter is positioned upstream from a chamber of the filtering device, wherein the filtering device further includes a filter assembly positioned downstream of the chamber, and wherein causing the filter to separate the blood includes causing the blood to pass through the filter, into the chamber, and through the filter assembly.
20 The method of example 19 wherein the filter assembly includes multiple filter stages, and wherein causing the filter to separate the blood further includes causing the blood to pass through the multiple filter stages of the filter assembly.
21. A clot treatment system, comprising:
a catheter;
a filtering device;
a pressure source; and
a connector including—
   first port configured to be coupled to the pressure source,
   second port configured to receive material aspirated from a patient via the catheter, and
   a third port configured to direct the aspirated material toward the filtering device,
wherein the pressure source is configured to (i) apply a first pressure to the first port to aspirate the material from the patient via the catheter and through the second port and (ii) apply a second pressure to the first port to direct the aspirated material through the third port toward the filtering device.
22. The clot treatment system of example 21 wherein the connector further includes a first fluid control device operably associated with the second port and a second fluid control device operably associated with the third port.
23. The clot treatment system of example 22 wherein the first fluid control device includes a first check valve and wherein the second fluid control device includes a second check valve.
24. The clot treatment system of example 22 or example 23 wherein:
in response to the first pressure, the first fluid control device is configured to be opened to allow the aspirated material to flow therethrough and the second fluid control device is configured to be closed to at least partially prevent the aspirated material from flowing therethrough; and
in response to the second pressure, the first fluid control device is configured to be closed to at least partially prevent the aspirated material from flowing therethrough and the second fluid control device is configured to be opened to allow the aspirated material to flow therethrough.
25. The clot treatment system of any of examples 22-24 wherein the connector further includes a y-shaped body that defines the first, second, and third ports and a first flow path extending between the first and second ports and a second flow path extending between the first and third ports.
26. A method of treating clot material within a patient, the method comprising:
applying, via a pressure source, a first pressure to a port of a connector to aspirate clot material into the pressure source via a catheter and along a first flow path defined by the connector, wherein the connector is located downstream from the catheter and upstream from a filtering device;
applying, via the pressure source, a second pressure to the port to move at least a portion of the aspirated clot material along a second flow path defined by the connector and into the filtering device; and
causing a filter of the filtering device to separate blood from the portion of the aspirated clot material.
27. The method of example 26 wherein causing the filter to separate the blood includes allowing gravity to drive the blood through the filter.
28. The method of example 26 wherein causing the filter to separate the blood includes creating a pressure gradient across the filter to drive the blood through the filter.
29. The method of example 28 wherein creating the pressure gradient includes applying a vacuum pressure to a downstream side of the filter.
30. The method of any of examples 26-29 wherein the filter is positioned upstream from a chamber of the filtering device, wherein the filtering device further includes a filter assembly positioned downstream of the chamber and having multiple filter stages, and wherein causing the filter to separate the blood includes causing the blood to pass through the filter, into the chamber, and through the multiple filter stages of the filter assembly.

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A filtering device, comprising:
a body defining a body chamber;
a lid coupled to the body and defining a lid chamber configured to receive material aspirated from a patient;
a filter tray configured to be positioned at least partially between the body chamber and the lid chamber and to permit a first portion of the material to pass from the lid chamber into the body chamber;
a filter assembly configured to receive the first portion of the material from the body chamber and permit a second portion of the material to pass therethrough; and
an outlet conduit fluidly coupled to the filter assembly and configured to allow the second portion of the material to be removed therefrom.

2. The filtering device of claim 1 wherein the material aspirated from the patient includes clot material from within a patient and wherein the first and second portions of the material include blood.

3. The filtering device of claim 1 wherein the filter assembly includes an annular filter with one or more filter stages and configured to direct the second portion of the material radially inwardly across the one or more filter stages.

4. The filtering device of claim 3 wherein the one or more filter stages includes a first filter stage having a first rating and a second filter stage having a second rating different than the first rating.

5. The filtering device of claim 4 wherein the first rating is at least 100 μm and wherein the second rating is up to 50 μm.

6. The filtering device of claim 1 wherein the lid includes a valve configured to control access to the lid chamber.

7. The filtering device of claim 6 wherein the valve includes a cross-slit valve.

8. The filtering device of claim 6 wherein the valve is configured to receive the material aspirated from the patient via a pressure source and without being operably coupled to the pressure source.

9. The filtering device of claim 6 wherein the valve is configured to receive the material aspirated from the patient via a syringe and without being operably coupled to the syringe.

10. The filtering device of claim 1, further comprising a fluid control device operably coupled to the outlet conduit and configured to be transitioned from (i) a closed state, in which the fluid control device inhibits or prevents the second portion of the material within the outlet conduit from flowing through the fluid control device, to (ii) an open state, in which the fluid control device allows the second portion of the material within the outlet conduit to flow through the fluid control device.

11. The filtering device of claim 1 wherein the body defines an opening to the body chamber and wherein the filter tray is positioned at least partially within the opening.

12. A method of treating clot material within a patient, the method comprising:
repeatedly aspirating clot material into a pressure source of a clot treatment system via a catheter of the clot treatment system;
after one or more of the aspirations, emptying at least a portion of the aspirated clot material contained within the pressure source onto a filter of a filtering device; and
causing the filter to separate blood from the portion of the aspirated clot material.

13. The method of claim 12 wherein causing the filter to separate the blood includes allowing gravity to drive the blood through the filter.

14. The method of claim 12 wherein causing the filter to separate the blood includes creating a pressure gradient across the filter to drive the blood through the filter.

15. The method of claim 14 wherein creating the pressure gradient includes applying a vacuum pressure to a downstream side of the filter.

16. The method of claim 14 wherein creating the pressure gradient includes applying a positive pressure to an upstream side of the filter.

17. The method of claim 12, further comprising:
removing the filtered blood from the filtering device, and
reinfusing the filtered blood into a patient.

18. The method of claim 17 wherein:
the filtering device includes an outlet conduit configured to contain the filtered blood and a fluid control device configured to control access to the filtered blood within the outlet conduit,
removing the filtered blood includes—
operably coupling a syringe or other vessel to the fluid control device, and
receiving the filtered blood within the syringe or other vessel; and
reinfusing the filtered blood includes reinfusing the filtered blood using the syringe or other vessel.

19. The method of claim 12 wherein the filter is positioned upstream from a chamber of the filtering device, wherein the filtering device further includes a filter assembly positioned downstream of the chamber, and wherein causing the filter to separate the blood includes causing the blood to pass through the filter, into the chamber, and through the filter assembly.

20. The method of claim 19 wherein the filter assembly includes multiple filter stages, and wherein causing the filter to separate the blood further includes causing the blood to pass through the multiple filter stages of the filter assembly.
